(19) United States
(12) Patent Application Publication
(10) Pub. No.: US 2007/0196439 A1
(43) Pub. Date: Aug. 23, 2007

(54) LACTOSE-REDUCED DAIRY COMPOSITIONS AND RELATED METHODS

(76) Inventors: Steven J. Catani, Athens, GA (US); Matthew Todd Robinson, Basel (CH)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/670,198

(22) Filed: Feb. 1, 2007

Related U.S. Application Data
(60) Provisional application No. 60/772,819, filed on Feb. 13, 2006.

(51) Int. Cl. A61K 35/74; A61K 47/00

(52) U.S. Cl. 424/439; 424/93.45

(57) ABSTRACT

Compositions for ameliorating the symptoms associated with lactase deficiency, the composition including a lactose reduced dairy product, and an effective amount of a probiotic, a prebiotic, or a mixture thereof. The lactose reduced dairy product is selected from a fluid milk, a smoothie, a liquado, ice cream, yogurt, and a yogurt drink. Methods for treating lactose intolerance in a patient in need thereof, the method includes providing a composition having a lactose reduced dairy product, and an effective amount of a probiotic, a prebiotic, or a mixture thereof. The lactose reduced dairy product is selected from a fluid milk, a smoothie, a liquado, ice cream, yogurt, and a yogurt drink.

### FIELD OF THE INVENTION

The present invention relates a lactose reduced dairy composition for reducing symptoms associated with lactase deficiency in humans and related methods. More particularly, the present invention relates to diary compositions, fluid or otherwise, that are symbiotic compositions or have either a prebiotic or a probiotic.

### BACKGROUND OF THE INVENTION

Lactose is a natural sugar found in fluid milk and milk products of all mammals. It is a disaccharide made of the monosaccharides glucose and a galactose. The lactase enzyme splits and hydrolyzes lactose into its component monosaccharides (i.e., glucose and galactose) for transport across the cell membrane. The lactase enzyme is naturally secreted in the small intestine. If lactase is not present, or not present in sufficient levels, lactose passes through the small intestines into the large intestine where it is fermented by a bacteria in the colon. Such fermentation produces hydrogen and methane. Because the colon (i.e., large intestine) has a limited capacity to eliminate gas formed by fermentation via absorption and respiration, quantities above that limited capacity cause bloating, gastric distention and pain, and flatulence.

Lactose intolerance is a natural condition that may affect up to 75 percent of the world's population and as many as 50 million, or 20 percent, of all people in the United States. The condition is particularly prevalent among certain ethnic and racial populations. For example, it is estimated to affect as many as 50 percent of Hispanics, 80 percent of African Americans, and 90 percent of Asian Americans. Lactose intolerance generally manifests itself by the time an individual reaches his or her 20s, although it is possible to develop dairy digestive problems at any age.

Individuals having lactose intolerance currently have several choices to avoid the uncomfortable symptoms associated with lactose intolerance. As described in more detail below, these include abstaining from non-fermented dairy products, taking a solid lactase supplement, and ingesting only lactose-free or lactose reduced-reduced products.

Abstaining from ingesting lactose-containing products, such as non-fermented dairy products, can eliminate the symptoms associated with lactose intolerance, it is far from ideal as non-fermented diary products are an important part of the modern diet.

Oral dosage forms useful for treating or controlling lactose intolerance have long been known. See, for example, U.S. Pat. No. 3,627,583. Using solid lactase tablets aids lactose intolerant people digest milk and milk products.

One potential drawback to solid lactase tablets is that it is known that an acidic environment, like that found in the stomach, inactivates or destroys typical lactase enzymes. In addition, enzymes exist in the gastro-intestinal tract, e.g., proteases, that break down or inactivate active enzymes, such as, lactase. Such destruction in activity reduces the efficacy and potency of solid oral dosage forms formulated using typical lactase enzyme preparations. Attempts to address this problem have focused on the lactase enzyme used, e.g., the environmental pH used to grow the lactase producing fungi or yeast.

Other attempts have focused on coating the lactase enzyme using coatings known to those skilled in the art to protect the enzyme during transit, only releasing it on arrival in the small intestine. While the coatings permit the lactase enzyme to get to the small intestine, their release parameters are highly dependent on local conditions in the gastro-intestinal pathway. If the enzyme is released too early it can be deactivated by the conditions in the stomach. If released too late it will not be in position to catalyze the breakdown of the lactose and prevent the adverse effect.

Even when the lactase enzyme is released from a coating at the proper time and location, the residence time of the enzyme in the small intestine is limited by the natural flow of material through the body. In practical terms this requires lactase enzyme to be ingested continually in order to provide a constant level, or alternatively, it can be consumed just prior to foods that may contain lactose. To be effective on a non-continuous basis the person using the lactase would be required to predict the lead time required to deliver and release the enzyme to the correct location.

Third, lactose reduced diary products are also available to consumers. One such product for sale in the United States is LACTAID brand lactose-reduced milk. This product is dairy milk that has its lactose content reduced by pre-hydrolysis of lactose using lactase enzyme. Other methods to reduce the lactose content of fluid dairy milk can also be used, e.g., ultrafiltration. Several lactose-reduced products are available for consumers to choose from including normal and reduced fat milks, yoghurts, cheeses, ice cream.

These products do not replace the lactase in the digestive track, but instead prevent the gastric discomfort by removing it from the food. The consumer can ingest the dairy product, and incur the benefits associated with its nutrient content without the downside of the lactose that would ferment in their colon and cause discomfort. While lactose-reduced products provide an effective solution they presume that a lactase deficient consumer has access to lactose-reduced version of all dairy products in all venues in which they eat. This is clearly not the case, especially for out of the home eating occasions, leaving a clear need for a different solution.

A more recent introduction is a product sold under the LACTAGEN brand. This product is apparently provides a systems-based approach to attempt to “teach” the body to digest dairy products. According to www.lactagen.com/about.do, the combination of taking yogurt with live cultures, having meals with the formula, taking specific dosages and with the combination of lactose, tricalcium phosphate, lactobacillus Acidophilus, fructo-oligosaccharides (FOS) and cellulose gum and silica, will “teach” the body to digest dairy products.

Those skilled in the art will appreciate adapting the gut flora to tolerate other sugars not readily digested in the small intestine. It is known that the tolerance to inulin, a fructo-oligosaccharide contained in garlic, onions, and the tubers of plants like chicory or Jerusalem Artichoke, can be increased by regular consumption. While not wishing to be bound by any particular theory, by shifting the colonic flora to acid forming bacteria, the rate of fermentation can be slowed, which is believed to permit greater consumption of gas forming foods. In the case of inulin, the fermentable substrate itself promotes the selective growth of acid forming bacteria. As these bacteria are promoted the individual can then ingest more inulin without gastric distress, which in turn further promotes the growth of the beneficial bacteria. While the effect builds on itself with continued small increases in consumption, it is also reversible in the same manner. That is if the consumer ceases to consume inulin the colonic flora can revert back to a state where little inulin can be tolerated. Further, the effect has an upper limit and most people, even those with well-adapted colonic flora, have a limit to how but inulin they can consumer.

In the case of lactose, the same is not true, lactose ingestion by itself does not selectively support acid forming bacteria. However, by routine consumption of acid forming bacteria, such as, various strains of lactobacillus and bifidus, an environment more favorable to slower gas production will be created, and hence less gastric issues when lactose is ingested. While this approach may be effective for small amounts of lactose ingestion, or for individuals with some level of endogenous lactase production, large amounts of lactose taken in a relatively short periods will still exceed the colon's ability to remove gas without pain or flatulence.

Better solutions are needed. The present invention is directed to such solutions. Herein we disclose a solution that overcomes all of the issues noted above and provides a lactase deficient consumer opportunity to enjoy dairy, including fluid milk, and other lactose containing products, reap their health and sensory benefits, and not be beset with gastric distress. Moreover it provides the ability to accomplish this in a way that is both economical and easily fits into the lactase deficient consumer's lifestyle. Finally, it provides a solution in which the cause of the problem becomes the source of the solution.

### DETAILED DESCRIPTION

A composition of the present invention relates to a lactose reduced dairy composition containing a prebiotic, probiotic, or mixtures thereof. It is believed that the composition of the present invention modifies the colonic flora in such a way as to increase the tolerance for fermentable carbohydrates. In addition, regular replenishment of the colonic flora with bacteria known to improve the ability of a person to tolerate fermentable carbohydrates is accomplished. This replenishment reserves the gas handling capacity of the colon for other challenges. Improving the gut flora increases the capacity for non-lactose reduced dairy. This change in gut and colon flora will result in the reduction of bloating, diarrhea, gastric distention and pain, and flatulence. Regular consumption of this new dairy composition improves the users ability to tolerate diary in general.

A prebiotic is a typically a carbohydrate. In the present invention, the prebiotic must be one that promotes the formation of a gut flora that slows fermentation. For example carbohydrate polymers of fructose, known as fructo-oligosacharides, are known to promote the growth of acid forming bacteria in the colon such as those in the genus lactobacillus or bifidus. Various oligosaccharides are classified as prebiotics and added to processed foods and supplements include galactan, carbohydrate based gums, such as, psyllium, guar, carrageen, gellan, konjac, FOS, neosugar, or inulin, fructo-inulins, lactitol, lactosucrose, lactulose, oligofructose, pyrodextrins, soy oligosaccharides, transgalacto-oligosaccharides (TOS), and xylo-oligosaccharides. The amount of prebiotic material used can be any effective amount. For example, fructo-oligosaccharides used from about 1 to about 5 grams per day have been shown to be effective. Three servings of dairy per day are recommended to assure adequate intake of calcium. A typical serving of dairy, if taken as fluid milk, is about 240 g. The daily dose of fiber can be divided into each serving of dairy. Typically the smallest effective dose is preferred. The prebiotic carbohydrate must be one that promotes the formation of a gut flora in which fermentation is slowed. For example carbohydrate polymers of fructose, known as fructo-oligosaccharides, or Neosugar, or inulin, are known to promote the growth of acid forming bacteria in the colon such as those in the genus lactobacillus or bifidus. Other polymers, such as, various galactans, and carbohydrate based gums, such as psyllium, guar, carrageen, gellan, konjac are also known to improve GI health. The carbohydrate Lactulose is also known to improve GI gas handling capacity.

Probiotic is commonly used to refer to “good” bacteria that one has to have in the body in order to maintain a healthy immune system. The probiotic can be any bacteria that increase the ability of the colon to slow the rate of fermentation. Typical bacteria in this class are those known to acidify the colon such as those from the genus lactobacillus or bifidobacteria. Lactobacillus acidophilus or bifidobacterium bifidum are known to provide this function. The amount of probiotic per serving can be any amount which provides for an effective flora in the colon. Probiotics of the present invention are ingested in an amount of from about 1×10⁶ to about 1×10⁹ colony forming units (cfu) per serving.

A composition containing both a prebiotic and a probiotic is known as a symbiotic product (probiotic +prebiotic =synbiotic).

Dairy products are an ideal carrier for probiotics because they are compatible with both carrying and preserving live bacteria, are generally distributed though refrigerated channels, and have a shelf life that allows for delivery of reliable quantities of bacteria. Other bacterial delivery-methods have to significantly over formulate bacteria levels so as to account for losses during shipment and while on the shelves in warehouses, store, and pantries.

While dairy is consumed throughout the day, it is a routine part of most breakfast allowing for ingestion of prebiotics and probiotics in a normal routine. Fluid dairy is easier to ingest than solid oral dosage forms, especially for people with swallowing problems. Other current solutions require adding a new routine to a consumer's lifestyle, something that is very difficult to do. Not providing for regular dosing of the pro or prebiotic agent will allow the colonic flora to return to the condition in which is has less capacity to handle lactose.

Lactose from milk makes up about 71% of that ingested by a typical consumer. Even just eliminating this single source of lactose reduces the load on the colon by and improves the ability of the optimized gut flora present to handle other lactose ingested. Just modifying the gut flora without removing the major sources of lactose from the diet may result in exceeding the gas handling capacity of the colon and result in gastric bloating, cramps, or flatulence.

An average person consumes about 21.9 g+/−0.5 g of lactose per day. Of this amount about 15.7 g+/−0.4 g is from milk, which leaves about 6.2 g+/−0.2 g from other sources. The average person consumes about 330 g+/−0.7 g of milk per day.

Lactose reduced milk typically has near 100% of the lactose removed. With the new compositions lower removal levels are possible. By increasing the ability of the colon to handle fermentable carbohydrates, the requirements to eliminate all lactose from the diet can be relaxed, allowing more flexible formulations and more economical production without loss of efficacy.

The dairy product can be any fluid milk or milk-based product. Such products include, chocolate milk, whole milk, fat-free milk, such as a smoothie, a liquado (a ready to drink fruit and dairy-based beverage where the dairy portion may be lactose free or a ready to drink fruit and water beverage. Both cases include forms that are similarly described as a shake, smoothie, or malt) or other dairy concoctions. It can be a fermented yogurt product or yogurt drink, cheese, ice cream, and the like based product. A composition of the present invention contains a lactose reduced dairy product, and an effective amount of a prebiotic, a probiotic, or both in order to improve the ability of a consumer to tolerate fermentable carbohydrates, e.g., lactose.

Dairy can come in multiple forms, including single serve packages proving consumers variety while not sacrificing convenience or requiring new routine.

The lactose reduced dairy product can be produced by any method know to those skilled in the art. For example, it can be produced by treating regular milk with lactase enzyme, hydrolyzing the lactose to it's constituent carbohydrates glucose and galactose. Alternatively. The lactose reduced dairy product can be produced by ultra filtration. Yet another method would use lactose as a substrate or donor for an enzymatic polymerization to a straight of branched carbohydrate polymer as disclosed in U.S. Pat. Nos. 5,952,205 and 6,423,833. The level of lactose reduction can be complete reduction, i.e., near 100%, or less.

A probiotic can be added to the diary product at any stage of its production, as long as the bacteria are not inactivated during processing.

In an embodiment, a prebiotic can be added to the lactose reduced product any stage of its production, as long as the prebiotic remains a viable energy source after processing.

In an embodiment the lactose reduced dairy product would contain both a probiotic and a prebiotic. In this embodiment, the prebiotic would help assure that the probiotic in the colon an energy source capable of sustaining or enhancing probiotic growth.

One embodiment of the present invention is dairy product, e.g., low fat, reduced fat or full fat milk, where the lactose is removed or reduced, ice cream, yogurt, cheese or cheese products or other dairy products potentially described as a one-shot, smoothie, shake, malt, creamer or other that delivers a probiotic and/or a bifidogenic or Lactobacillogenic-prebiotic that delivers about 1 to about 15 g of prebiotic per serving, where the prebiotic includes but is not limited to lactulose, short and long chain inulin, short and long chain fructo-oligosacharides, gallacto-oligosacharides, or partially hydrolyzed guar gum per serving or where the prebiotic is from about 0.5 to about 20% w/w of the final formulation and the examples of prebiotic are the same as listed above. Lower amounts, for example 0.5 grams per serving or 0. 1 grams per serving can also be used where the prebiotic is especially effective at supporting beificial bacteria over less desirable floras. What is important is slowing the rate of fermentation.

The examples contained herein are not intended to be limiting, but merely illustrative of the forms in which the invention may be used as part of a method for reducing symptoms associated with lactose intolerance in humans.

### EXAMPLE 1

Lactose reduced milk is fortified with probiotic as follows:

To a half-gallon of lactose free 2% milk (LACTAID® brand) about 8×10⁹ cfu's of Lactobacillus acidophilus are added, which represents about 1×10⁹ cfu's per serving.

### EXAMPLE 2

Lactose reduced milk is fortified with probiotic.

To a half-gallon of lactose free 2% milk (LACTAID® brand) about 1.×10⁹ cfu's of Bifidobacterium longum are added, which represents about 0.2×10⁹ cfu's per serving or about 0.6×10⁹ cfu's per day if three servings of milk are consumed per the National Dairy Council recommendation.

### EXAMPLE 3

Lactose reduced milk is fortified with probiotic.

To a half -gallon of lactose free 2% milk (LACTAID® brand) 66×10⁶ cfu's of Bifidobacterium bifidum are added, which represents about 8.3×10⁶ cfu's per serving or about 25×10⁶ cfu's per day if three servings of milk are consumed per the National Dairy Council recommendation.

### EXAMPLE 4

Lactose reduced milk is fortified with prebiotic.

To a half -gallon of lactose free 2% milk (LACTAID® brand) about 8 g of fructo-oligosaccharide is added. This represents about 1 g per serving.

### EXAMPLE 5

Lactose reduced milk is fortified with prebiotic.

To a half-gallon of lactose free 2% milk (LACTAID® brand) about 2.6 g of fructo-oligosaccharide is added. This represents about 0.33 g per serving or about 1 g per day if three servings of milk are consumed per the National Dairy Council recommendation.

### EXAMPLE 6

Lactose reduced milk is fortified with symbiotic.

To a half-gallon of lactose free 2% milk (LACTAID® brand) about 2.6 grams of fructo-oligosaccharide and about 2.6×10⁹ cfu's of Lactobacillus acidophilus is added. This represents about 0.33 gram per serving of fructo-oligosaccharide and about 333×10⁶ cfu's of Lactobacillus acidophilus per serving and about 1 gram of fructo-oligosaccharide and about 1×10⁹ cfu's of Lactobacillus acidophilus per day if three servings of milk are consumed per the National Dairy Council recommendation.

### EXAMPLE 7

A reduced lactose dairy product was prepared with the following ingredients: fat-free Milk, water, strawberry puree, sugar, cream, modified corn starch, carrageenan, pectin, cultured dextrose, natural and artificial flavors, potasium sorbate, lactase, malic acid, colors, vitamins A palmitate, and D2. The formulation had the following nutritional profile:

| | % RDI | Amount | |
|---|---|---|---|
| saturated fat | 5% | 3.5 | g |
| trans fat | 0% | 0 | g |
| cholesterol | 5% | 15 | mg |
| sodium | 4% | 100 | mg |
| total carbohydrates | 14% | | |
| dietary fiber | 2% | 0.5 | g |
| sugars | | 35 | g |
| protien | 12% | 6 | g |
| vitamin A | 15% | | |
| vitamin C | 10% | | |
| vitamin D | 30% | | |
| calcium | 20% | | |

The formula contained about 0.5 g per serving of pre-biotic fibers

### EXAMPLE 8

A reduced lactose dairy product was prepared with the following ingredients: Fat Free Milk, Water, Mango Puree, Sugar, Cream, Modified Corn Starch, Carrageenan, Pectin, Citrus Pulp, Cultured Dextrose, Natural and Artificial Flavors, Potasium Sorbate, Lactase Enzyme, Malic Acid, Colors, Vitamins A Palmitate, and D2. The formulation had the following nutritional profile:

Total Size: 8 oz Calories: 180

| | % RDI | Amount | |
|---|---|---|---|
| saturated fat | 9% | 1.5 | g |
| trans fat | 0% | 0 | g |
| cholesterol | 4% | 10 | mg |
| sodium | 3% | 80 | mg |
| total carbohydrates | 12% | | |
| dietary fiber | 1% | 0.25 | g |
| sugars | | 29 | g |
| protien | 10% | 5 | g |
| vitamin A | 10% | | |
| vitamin C | 6% | | |
| vitamin D | 25% | | |
| calcium | 15% | | |

The formula contained about 0.25 g per serving of pre-biotic fibers.

Although the invention is illustrated and described above with reference to specific embodiments, the invention is not intended to be limited to the details shown. Rather various modifications may be made in the details within the scope and range of equivalents of the components and steps without departing from the invention

1. A composition for ameliorating the symptoms associated with lactose deficiency, the composition comprising:
a lactose reduced dairy product, and
an effective amount of a probiotic, a prebiotic, or a mixture thereof.

2. A composition of claim 1, wherein the lactose reduced dairy product is selected from a member of the group consisting of a fluid milk, a smoothie, a liquado, ice cream, yogurt, and a yogurt drink.

3. A composition of claim 1, wherein the prebiotic is a carbohydrate polymer.

4. A composition of claim 3, wherein the carbohydrate polymer is selected from the group consisting of a fructan, a galactan, a food gum, a fructo-oligosaccharide, psyllium, lactulose, guar, carrageenan, mixtures thereof.

5. A composition of claim 4, wherein the carbohydrate polymer is present in an amount of from about 0.1 g to about 15 g per 240 g serving.

6. A composition of claim 4, wherein the carbohydrate polymer is about 0.33 g per 240 g serving.

7. A composition of claim 1, wherein the probiotic is selected from the group consisting of a member of the genus lactobacillus, the genus bifidobacteria, and mixtures thereof.

8. A composition of claim 1, wherein the probiotic is present in an amount of from about 1×10⁶ g to about 1×10⁹ cfu's per 240 g serving.

9. A composition of claim 8, wherein the probiotic is present in an amount of about 10×10⁶ g to about 0.5×10⁹ cfu's per 240 g serving.

10. A composition of claim 9, wherein the probiotic is present in an amount of about 0.3×10⁹ cfu's per 240 g serving.

11. A method for treating lactose intolerance in a patient in need thereof, the method comprising:
providing a composition comprising
a lactose reduced dairy product, and
an effective amount of a probiotic, a prebiotic, or a mixture thereof.

12. A method of claim 11, wherein the lactose reduced dairy product is selected from a member of the group consisting of a fluid milk, a smoothie, a liquado, ice cream, yogurt, and a yogurt drink.
